(19) United States
(12) Patent Application Publication
(10) Pub. No.: US 2026/0000478 A1
(43) Pub. Date: Jan. 1, 2026

(54) CONTROLLING SURGICAL VISUALIZATION SYSTEMS USING MULTI-MODAL USER UTTERANCES

(71) Applicant: Carl Zeiss Meditec AG, Jena (DE)

(72) Inventor: Fang YOU, Aalen (DE)

(21) Appl. No.: 19/246,944

(22) Filed: Jun. 24, 2025

(30) Foreign Application Priority Data
Jul. 1, 2024 (DE) 10 2024 118 626.7

(51) Int. Cl. A61B 90/00; G06F 3/01; G06F 3/038; G06F 3/16; G10L 15/22

(52) U.S. Cl. CPC A61B 90/361; G06F 3/013; G06F 3/015; G06F 3/017; G06F 3/038; G06F 3/167; G10L 15/22; A61B 2090/372; G10L 2015/223

(57) ABSTRACT

Provision is made for a computer-implemented method for controlling a surgical visualization system. A first user utterance of a first user utterance type is received, wherein this first user utterance extends over a time interval within a defined period of time. A multiplicity of second user utterances of at least one different second user utterance type are received, these being captured in a manner distributed within said period of time and varying within the period of time. The surgical visualization system is controlled using the first user utterance and at least one second user utterance that is prioritized based on temporal relationships of the second user utterances in relation to the time interval.

### TECHNICAL FIELD

Various examples of the disclosure relate to aspects for controlling a surgical visualization system, for example a visualization system comprising a surgical microscope. Various examples relate in particular to the processing of multi-modal user utterances that take place over different durations, such as for example linguistic commands, on the one hand, and eye movements, on the other hand, for controlling a surgical visualization system.

### BACKGROUND

In surgical environments, precise and reliable control of surgical visualization systems, for example optical surgical microscopes, is essential. Some systems use for example eye tracking and voice commands for control. However, noise in the eye-tracking data and delays in speech recognition often lead to inaccurate control processes. These inaccuracies may impair the reliability and accuracy of the surgical display systems.

### SUMMARY

There is therefore a need for improved techniques for controlling surgical visualization systems that overcome or alleviate at least some of the abovementioned limitations and disadvantages.

This object is achieved by the features of the independent patent claims. The dependent patent claims define further advantageous embodiments.

The solution according to the invention is described below both with reference to the claimed methods for controlling surgical visualization systems and with reference to the corresponding control devices and surgical visualization systems. Provision is furthermore made for corresponding computer programs and electronically readable storage media. It should be understood that features, advantages and alternative exemplary embodiments may be assigned to the respective other categories, and vice versa. By way of example, the control devices or surgical visualization systems may be improved by features used as part of the described methods for controlling surgical visualization systems, and vice versa.

Provision is made for a computer-implemented method for controlling a surgical visualization system. A surgical visualization system comprises for example a visualization system that is used in surgical environments or in a surgical application to present visual information to a user.

A surgical visualization system may be used in connection with endoscopic, microscopic or any other imaging methods or examination methods. Such imaging systems are used for example in surgical environments to present, for example to present in real time, visual information relating to a surgical operating region and/or a surgical sequence to a user. In particular, the visual information may be provided for a field of view of the surgical visualization system.

The visual information may comprise for example an image, multiple images and/or a video stream that may be based on 2D or 3D image data, and may furthermore comprise a presentation of a target region (region of interest, ROI) in an operating region.

The visual information, for example the operating region and possibly a surgical instrument, may be presented to the user for example using one or more of the following devices: a purely optical system (for example comprising an eyepiece) for optical presentations, a presentation on one or more electronic display devices (for example a display or screen) based on acquired image data, a presentation in a virtual reality system (for example a virtual display in a virtual environment), a presentation provided by a 3D display device that visually represents depth information for the user, or a presentation in an augmented reality system that projects virtual elements into real space. These various presentation devices make it possible to present and make the visual information accessible to the user.

Generally speaking, a surgical visualization system may thus comprise a combination of hardware and software components that acquires imaging data from examination devices such as endoscopic cameras, microscopes or other imaging devices and visually presents these image data to the user (in real time). These systems assist the user by providing clear and detailed visual information, in particular portions of the imaging data. By way of example, it is possible to present additional visual information regarding the real operating region, such as for example virtual instructions or navigation aids that assist the user. Other examples comprise for example text, descriptions, icons, superposition, etc. These may be overlaid, for example optically or virtually.

The method for controlling the surgical visualization system comprises the following steps:

In one step, a first user utterance is obtained. The obtaining may comprise capturing or detecting carried out using one or more first sensors. The first user utterance may be obtained in the form of raw data from a sensor or sensor data that are able to be processed. It would be conceivable for the first user utterance to be transmitted electronically, for example based on an external sensor measurement.

The first user utterance may be obtained in real time. A latency between the first user utterance and obtaining corresponding data indicating the user utterance may for example be shorter than 0.5 seconds, optionally shorter than 0.05 seconds, again optionally shorter than 0.005 seconds.

The first user utterance may comprise an action or instruction for a user, or generally information regarding a user using the surgical visualization system to present visual content. The first user utterance may be captured in order to control the surgical visualization system.

The first user utterance may comprise an intentional, but possibly also an unintentional, action that extends over a time interval of a period of time, in particular over the entire duration of the time interval.

In some examples, the first user utterance may comprise one or more of the following: a linguistic utterance made by the user, a gesture made by a body part of the user, a touch gesture on a touch interface, a brain/computer interface (BCI) signal, or a multimodal combination of the above.

The first user utterance may allow the user to express a user intention or user intent. By way of example, the first user utterance may serve to determine what type of control action the user expects from the surgical visualization system.

The first user utterance corresponds to a first user utterance type, in other words a first (user utterance) modality. A user utterance type may refer to a category or type of user action performed by the user.

Examples of first user utterances that may express a user intention are linguistic utterances from which it is possible to use processing to determine voice commands such as “enlarge this region”, “focus on this structure”, “zoom in here”, or “show this as an overview”. These linguistic utterances may indicate what presentation change the user wants. Gestures made by at least one body part of the user, for example in order to define an image section, may also express a user intention. As a further example, it would be possible for example to use gestures that are performed by way of an instrument or tool, for example a surgical tool. By way of example, it would be possible to use a pointer instrument to define the first user utterance.

Gestures made by at least one body part of the user may take various forms and incorporate a multiplicity of body parts. Hand gestures are a common example in which the user moves or positions their hands or fingers in a certain way to indicate an intention or action. By way of example, a particular body part or one or more instruments could be moved into a particular region or into a particular zone and then stay there. By way of example, hand movements or finger movements in certain shapes or patterns would be conceivable. Blinking patterns or gestures on a touchscreen are also conceivable.

The first user utterance may serve to determine, for the surgical visualization system, a first user input used to specify an action, in particular a control action, of the surgical visualization system. The first user utterance may be processed in order to determine therefrom for example a desired control action for the surgical visualization system. In this case, the processing of a user utterance for determining a user input may be carried out for example in a rule-based manner, or may be based on machine learning.

A first user input may be determined based on the first user utterance. The user input may comprise, for example, a user intention. The user input may comprise, for example, a control command for a desired control action for the surgical visualization system.

The first user utterance extends over a time interval. A time interval refers to a particular time period during which the user utterance takes place. The first user utterance thus takes place throughout the entire time interval; it extends over the entire time interval, that is to say from the start to the end of the time interval. The first user utterance is accordingly captured by the surgical visualization system over the entire time interval, that is to say from the start to the end of the time interval, using sensors.

Time information characterizing the time interval, for example a start time (point in time) and/or an end time (point int time) and/or a midpoint (point in time) of the time interval, may additionally be acquired and stored. By way of example, the start time indicates a start of the time interval, the end time indicates an end of the time interval and the midpoint indicates a middle of the time interval. The start time, the end time and the midpoint are exemplary reference times of the time interval. Furthermore, a length of the time interval may also be acquired and stored as time information.

The time interval lies in a longer period of time, that is to say is contained in the period of time. The period of time comprises the time interval. The period of time may have the same length as the time interval or be longer.

In another step, a multiplicity of different second user utterances are obtained, for example captured or detected, these being assigned to at least one second user utterance type. It is possible for the second user utterances to be transmitted electronically from at least one second sensor.

The designation “first user utterance” and “multiplicity of second user utterances” is not intended to imply any order or hierarchy between the first and second user utterances. By way of example, it would be conceivable for the multiplicity of second user utterances to be obtained before the first user utterance.

Each second user utterance may be obtained in real time. A latency between the respective second user utterance and obtaining corresponding data indicating this second user utterance may for example be shorter than 0.5 seconds, optionally shorter than 0.05 seconds, again optionally shorter than 0.005 seconds.

Each of the multiplicity of second user utterances is associated with (respective) time information. The time information may be indicative of a time (point in time) or a duration at which or for which the second user utterance took place. Such time information is also obtained. In principle, the time information may be obtained implicitly or explicitly. By way of example, timestamps could be obtained. However, it would also be conceivable to infer the respective times at which a particular second user utterance took place based on a sampling rate and the sequence of the second user utterances.

The time information may in particular indicate how the respective second user utterance is arranged in relation to the time interval over which the first user utterance extends. A temporal relationship between each of the second user utterances and the first user utterance may thus be determined on the basis of the time information and the time interval.

The second user utterances are thus in a temporal correlation with the first user utterance, as may be indicated by the temporal relationship. By way of example, there may be an intentional or content-based correlation between the first and second user utterances that is represented by the temporal relationship. By way of example, the first and second user utterances may be in a common action or interaction context of a user/system interaction that is represented by the temporal relationship. By way of example, the first and second user utterances may be jointly correlated with a user intention.

The method may comprise determining the temporal relationship between each of the multiplicity of second user utterances and the first user utterance that extends over the time interval. In this case, the temporal relationship is determined based on the time information available for each of the second user utterances.

The second user utterances of the multiplicity of second user utterances are buffered. In particular, it is possible for the second user utterances to be buffered until it is possible, based on the time information for the second user utterances, to determine temporal relationships between each of the multiplicity of second user utterances and the time interval over which the first user utterance extends. By way of example, it would be conceivable for one or more second user utterances to be obtained before an end of the time interval; then, at least in some examples, it may not yet definitively be possible to determine the temporal relationship thereof in relation to the time interval (which has not yet ended or the end of which will take place at an as yet unknown future time). Accordingly, it is then possible to buffer the second user utterances until it is ultimately possible to determine temporal relationships between each of the multiplicity of second user utterances and the time interval. Furthermore, it is possible for the second user utterances to be buffered for at most or exactly a predefined or determinable buffer duration.

In some examples, the second user utterances and their time information may each be buffered in association with one another for a certain buffer duration in a buffer data structure (buffer). The buffer data structure may comprise for example a FIFO (first-in-first-out) memory. The size of the buffer or the buffer duration for which data are held may in this case be predefined, or may be determined for example based on a first user utterance type of the first user utterance. The buffer duration may be in the form of a sliding time window. The buffer duration may in particular be selected to be greater than a permissible maximum value for the time interval of the first user utterance.

Next, aspects in connection with the second user utterances will be described. The at least one second user utterance type of the second user utterances is different from the user utterance type of the first user utterance.

The second user utterances are distributed within the (larger) period of time. In other words: The multiplicity of second user utterances are distributed within the period of time. This thus means that the second user utterances take place in a manner distributed at different times within the (larger) period of time and are expediently captured. The time information associated with the second user utterances indicates how the second user utterances are distributed over the period of time.

The second user utterances comprise different second user utterances of a respective one of the at least one second user utterance type.

The second user utterances may be captured by way of at least one second sensor or multiple second sensors, for example using sensor fusion, in order to control the surgical visualization system. The at least one second sensor may be different from the first sensor.

However, it would also be conceivable for the at least one second sensor to comprise the at least one first sensor, such that the at least one first sensor may also be used to capture the second user utterances. The second user utterance may also comprise a processed signal/processed sensor data from one or more sensors.

The second user utterances may comprise multiple different actions or states of a user, that is to say different items of information regarding the user, which occur in a manner scattered at different times (point in times) over the period of time. The second user utterances take place several times over the period of time, that is to say at different times. The multiplicity of second user utterances comprises different (that is to say varying) second user utterances of the same second user utterance type in order to control the surgical visualization system.

By way of example, a plurality of the second user utterances may fall within the time interval over which the first user utterance extends. These second user utterances, which lie within the time interval over which the first user utterance extends, may vary among one another.

In some examples, the second user utterances may serve to specify a target point, or a target region, in the image data for the user intention of the first user utterance.

By way of example, the second user utterances may comprise pointing the user towards a target region. The target region may be arranged in the field of view of the surgical visualization system. The field of view may be a presented or actual field of view of the surgical visualization system. By way of example, the user could point to a presentation of the target region in an image captured by way of the surgical visualization system. However, it would also be possible for the user to point directly toward the target region, for example using a finger or a surgical instrument, or instrument for short. In the latter case, the pointing may be observed in an image captured by way of the surgical visualization system.

The second user utterances may comprise both intentional and unintentional actions or states of the user, such as for example gazes, body postures or body orientations, positions and/or orientations of at least one body part of the user (for example in a coordinate system of the surgical visualization system, or in particular in a field of view of the surgical visualization system), touching of a mechanical interface or a touch interface, or other information of the user that is acquired momentarily using sensors. The second user utterances serve to determine, for the surgical visualization system, second user inputs on the basis of which it triggers or parameterizes for example a control action that was determined by the first user utterance.

By way of example, it would be conceivable for the second user utterance to indicate a gaze direction of the user. A gaze direction of the user may be determined for example by pupil tracking (also referred to as eye tracking). It would thus be conceivable for the gaze direction to be determined at a particular sampling rate, wherein the sampling rate is selected to be high enough also to determine rapid changes in gaze direction. By way of example, it would be conceivable for the sampling rate used to determine the gaze direction to be in the range of not less than 100 Hz or optionally not less than 200 Hz. It is thereby possible to capture even rapid, jerky eye movements by way of which the eyes are moved between two points in order to realign the field of view. By way of example, such movements—which are physiologically induced—typically take place within 20 to 40 ms. A large number of second user utterances are accordingly obtained if the gaze direction of a person is determined in each case at a correspondingly high sampling rate. This should be understood to mean that it is not necessary for each second user utterance also to specify a different user input, that is to say for example a different gaze direction of the eye. In other words, different second user utterances of the multiplicity of second user utterances may be the same or at least comparable (for example if the eyes gaze in one direction for longer).

In preferred examples, the first user utterance may comprise a linguistic utterance made by the user, and the second user utterances may comprise at least one first group of second user utterances comprising gaze directions of the user, and comprise at least one second group of second user utterances comprising a position and/or orientation of a surgical instrument operated by the user. It is possible to determine second user inputs with higher accuracy from such a multi-modal combination of second user utterances of different second user utterance types.

The second user utterances may be captured at certain times, or at a predetermined sampling rate, such that different second user utterances are present, or at least may be present, at these times within the period of time. The sampling rate may make it possible to determine the time information for each second user utterance.

As already described above, different second user utterances (having different time information) may be different from one another or identical. In any case, it is thereby possible to obtain a temporal sequence of second user utterances of the same second user utterance type that were captured at the different times.

The second user utterances may provide additional parameters or context information for control based on the first user utterance.

The second user utterances may specify for example target locations, target regions, directions or selection regions in connection with a control action for the presentation of the surgical visualization system.

By way of example, the second user utterances may comprise a gaze direction or the gaze position of the user, which may easily be converted among one another, for example in relation to a presentation of the surgical visualization system or another reference coordinate system (for instance defined by a presented or actual field of view of the surgical visualization system).

The position and/or orientation of the head or part of the head of the user may also be captured as a second user utterance of a further second user utterance type. Likewise, a position and/or orientation of a body part, for example of at least one finger of the user, may be captured as a second user utterance.

A further example of a second user utterance may comprise a position and/or orientation of an instrument at a particular time. By way of example, the position and/or orientation of the surgical instrument may be determined in relation to the field of view of the surgical visualization system or in relation to the operating region. Furthermore, the position of a surgical instrument, or of at least part, for example the tip, of a surgical instrument may be determined in the machine coordinate system, that is to say in 3D world coordinates, or another reference coordinate system, for example using sensors. Such a position of a surgical instrument may also be determined for example from image data from an imaging system of the surgical visualization system, said image data imaging at least the part of the surgical instrument.

The second user utterances may be shorter relative to the first user utterance. The second user utterances may be captured more frequently relative to the first user utterance. Thus, for example, a time difference between two successive second user utterances may be less than 30%, or less than 20%, or less than 10%, or less than 5%, or less than 1% of the time interval over which the first user utterance extends. Capturing a (optionally each) second user utterance may require in each case less time compared to capturing the first user utterance. By way of example, less than 50%, or 30%, or 20%, or 10%, or 5%, or 1% of the time required to capture the first user utterance. The second user utterances may be recorded at a higher temporal density than the first user utterance. The second user utterances may be less complex than the first user utterance. The second user utterances may be recorded at a higher temporal density than the first user utterance. The second user utterances may be captured with a shorter time difference from one another than the duration of the first user utterance. They supplement the user intention determined based on the first user utterance in order to provide details for precise control of the visualization system. The second user utterances may also be used to determine the first user input. By way of example, they may serve as continuous feedback mechanisms that enable the system to respond to dynamic changes in the behaviour of the user in response to the determination and performance of the control action.

In some examples, each of the second user utterances has its own temporal extent defined by a respective duration. In this case, each of these durations is shorter than the first-mentioned time interval over which the first user utterance extends. Thus, each considered individually, the second user utterances have a shorter duration than the first user utterance. The respective durations of extent of the second user utterances may be represented by the time information or timestamps associated with the respective second user utterances.

Preferably, a length of each of the durations may be less than 50% of the length of the first-mentioned time interval, particularly preferably less than 10%.

The second user utterances may serve to provide spatial and/or temporal parameters for a control action. Associated second user inputs may be determined for one or more second user utterances. By way of example, it would be conceivable for an associated second user input to be determined for each second user utterance of the multiplicity of second user utterances and for example to subsequently be buffered.

In some examples, each second user input may be determined from second user utterances of more than one, more than two, or more than three different second user utterance types.

By way of example, both a gaze direction and an instrument position may be processed together (as second user inputs of different user utterance types) in order to determine a target position of the user (user input).

The second user utterances and/or user inputs may supplement or characterize the first user input. The second user utterances and/or user inputs may parameterize an action, in particular a control command or a control action, of the surgical visualization system.

By way of example, the at least one second user input may comprise temporal control parameters for the control, for instance control the timing of the sequence of a control action of the surgical visualization system, for example define the start or the end of the control action, or parameterize and/or trigger individual phases of the control action.

The method further comprises prioritizing at least one second user utterance from the multiplicity of second user utterances based on the temporal relationships. Subsequently, the surgical visualization system may then be controlled based on the first user utterances and the prioritized at least one second user utterance. In particular, it would be possible for the surgical visualization system to be controlled based on at least one second user input determined based on the prioritized at least one second user utterance.

Such prioritizing of the at least one second user utterance may comprise selecting the at least one second user utterance. This means that only the at least one second user utterance is taken into account when controlling the surgical visualization system; however, those second user utterances that are not selected are not taken into account when controlling the surgical visualization system. However, it would also be conceivable for such prioritizing of the at least one second user utterance to comprise weighting the corresponding second user utterance to a greater extent when controlling the surgical visualization system compared to non-prioritized second user utterances. By way of example, user inputs may be determined for the various second user utterances; for example, such user inputs could indicate a particular position. A weighted average of such positions could then be determined.

The temporal relationships between all second user utterances may thus be used to perform the control, wherein either at least one of the second user utterances is used for the control based on their temporal relationship, and/or a different control parameter is determined based on the temporal relationship between the second user utterances and/or multiple (for example all) second user utterances are weighted differently for the control, and/or particular second user utterances are excluded for the control based on their temporal relationship.

Since the at least one second user utterance is prioritized based on the temporal relationship, it is possible to avoid an unintentional second user utterance being taken into account for the control of the surgical visualization system during a relatively long time interval over which the first user utterance extends. Unintentional control actions are thereby avoided. In detail, various techniques described herein are based on the finding that, in the case of relatively long time intervals over which the first user utterance extends—this is typically the case with a speech input, which may extend over a few seconds—some users may have difficulties in continuously and/or consistently maintaining uniform second user utterances that take place on a shorter time scale. One example would be the combination of a speech input (first user utterance) with a gaze direction (second user utterances), for example. During the speech input, which may for example last over 3 to 5 seconds, the user may be caused to change their gaze direction briefly. The gaze direction may be captured at a sampling rate of 100 Hz, such that the short change in gaze direction expresses itself as different second user utterances during the time interval. Since the temporal relationship between the various second user utterances and the first user utterance is taken into account, it is possible for example to take into account in particular those second user utterances that are arranged at the start or shortly before the end or at the end of the first user utterance. Other second user utterances, which take place for example at the start or in the middle of the time interval, might not be taken into account, or may be taken into account only with lower weighting. Analogously, the prioritization may also take place for other temporal relationships.

A temporal relationship may in each case represent a temporal correlation between the first user utterance and the respective second user utterance. The temporal relationship may comprise information indicating the temporal sequence or temporal arrangement of the user utterances in relation to one another. In some examples, the temporal relationship indicates the time difference with which the respective second user utterance was captured relative to the first user utterance or in particular a reference time (for example the end point or the midpoint) of the time interval over which the first user utterance extends.

A temporal relationship may be defined for example as a time difference between the (first-mentioned) time interval over which the first user utterance extends or a first (reference) time that is assigned to this time interval and/or to the first user utterance, and a second time or second time interval that is assigned to the at least one second user utterance. This time difference may be positive, for example, if the second user utterance is made after the first, or negative if the second user utterance is before the first. Therefore, for example, the times of the respective second user utterances, or second user inputs, may be in a temporal relationship with the time interval of the first user utterance.

The temporal relationship may also describe whether the second user utterance was captured during the time interval, or at least partially during the time interval. In this case, the second user utterance takes place completely or partially within the time interval in which the first user utterance also takes place. The temporal relationship may thus for example indicate whether the first and the at least one second user utterance take place at least partially in parallel.

By way of example, the temporal relationship may also be characterized by a predetermined time difference or threshold value. This may indicate the permitted maximum or minimum size of the time difference between the first and second user utterance for the second user utterance to be considered to be relevant to the control.

It is then possible for a check to be performed for each second user utterance. It is thus possible to check whether the respective temporal relationship, which is determined for the corresponding second user utterance, satisfies one or more predefined check criteria. The at least one second user utterance, which is subsequently taken into account when controlling the surgical visualization system, may then be selected depending on a result of these checks performed for all second user utterances. In other words, it is possible for example to select those second user utterances for which the check has a positive result.

Examples of such a check are described below. By way of example, it could be stipulated that only second user utterances that are made before the end of the first user utterance are taken into account. Or it would be possible to use only those second user utterances that lie at most 1 second or at most a certain decimal multiple, for example 1.5 times or twice, the length of the time interval over which the first user utterance extends, before the start of the first user utterance.

Since the second user utterances are buffered together with the correspondingly associated time information, this achieves flexibility in terms of retrospectively determining and checking the temporal relationships for the various second user utterances after the first user utterance has finished—that is to say the time interval has finished. It is thereby also possible to evaluate temporal relationships in relation to one or more check criteria relating to the end of the time interval—or generally to a reference time that then establishes for the first time when the time interval has ended. It is thus possible to use reference times determined by the end of the time interval to define a specific time range. It is then possible to check whether or not a time associated with a particular second user utterance lies in this time range.

A further advantage in connection with the buffering of the second user utterances together with the time information concerns the possibility of further processing the first and/or second user utterances, for example in order to determine user inputs for one or more of the first and/or second user utterances. By way of example, speech recognition could require a certain amount of time. Such further processing may take a certain amount of time due to limited computational resources. This latency may, without buffering, lead to distortions when determining the temporal relationships; such distortions are able to be avoided thanks to the buffering.

By way of example, it would in particular be possible to prioritize or in particular select those second user utterances that lie in a particular time period defined starting from the end of the time interval. Time periods starting from the middle of the time interval could also be taken into account. By way of example, it would be possible to prioritize or in particular select those second user utterances that extend within the last 20 ms of the time interval or within a time period that extends from the middle of the time interval 50 ms toward the start of the time interval. Furthermore, the one or more check criteria may comprise a check to determine whether a time associated with a corresponding second user utterance and indicated by the time information lies in a particular time range before the end of the time interval over which the first user utterance extends.

It is also possible to check whether a time associated with a corresponding second user utterance and indicated by the time information lies at most at the end of the time interval over which the first user utterance extends.

Usually, determining the first user input based on the first user utterance requires a certain amount of time, for example due to limited computational resources. The time when the first user input is determined may thus lie after the end of the time interval over which the first user utterance extends. Using the test criterion, for example, it is possible to ignore those second user utterances that lie for example starting from or after the end of the time interval over which the first user utterance extends.

Generally speaking, taking into account the temporal relationships and temporal check criteria, that is to say determining temporal threshold values or valid time windows or time ranges relative to the time interval, makes it possible to restrict the search space for relevant second user utterances. This allows more targeted selection of second user utterances that are in a relevant temporal correlation with the first user utterance.

In some variants, further check criteria could also be checked. By way of example, a number of the second user utterances that are supposed to lie before, during or after the time interval of the first user utterance. By way of example, there could thus be a requirement for a predetermined percentage, for example at least 50%, or a predetermined number of the second user utterances to lie within the time interval. By way of example, there could also be a requirement for a time difference (sampling rate) between the second user utterances that lie in the time interval of the first user utterance not to be fallen below. Instead of the time interval of the first user utterance, it is also possible for example to use in each case, as a reference, a time interval of equivalent length that may for example be shifted in a predefined manner in relation to the time interval of the first user utterance, wherein said time interval lies in particular at least partially, optionally completely, before the end of the time interval of the first user utterance.

By way of example, the second user utterances could be weighted (as a form of prioritization) depending on their temporal position relative to the first user utterance. In other words, a weighting of the second user utterances could depend on the temporal relationship between the respective second user utterance and the first user utterance. By way of example, those second user utterances that lie closer to the start or in the middle or at the end of the first user utterance could be weighted to a greater extent. In particular, a weighting of the second user utterances could depend on whether the respective second user utterance lies closer to a start or closer to the middle or closer to the end of the time interval of the first user utterance.

By way of example, it is possible to select that second user utterance that lies closest in time to the start or the middle or the end of the first user utterance.

By way of example, it is possible to select contiguous sequences of second user utterances that have certain temporal patterns relative to the first user utterance. By way of example, it would be possible to search for a sequence that starts shortly before the first user utterance and ends shortly thereafter.

Taking into account the temporal relationships, in particular specifying suitable (temporal) check criteria, allows the system to prioritize those second user utterances that are in a relevant temporal correlation with the first user utterance and are thus probably relevant to the control. The exact selection of the temporal criteria may in this case depend on factors such as the type of the first user utterance, the application context or user preferences. It is thereby possible for example to avoid an unintentional second user utterance being taken into account for the control of the surgical visualization system during a relatively long time interval over which the first user utterance extends. Unintentional control actions are thereby avoided.

Taking into account and checking the temporal relationships between the first user utterance and the second user utterances when controlling the surgical visualization system makes it possible to incorporate the temporal sequence of the user utterances into the control. It is thereby possible to improve the interaction between user and system since the temporal context of the user utterances is taken into account.

As an alternative or in addition to the temporal relationship between the first user utterance and the second user utterances, it is also possible to take into account temporal relationships between the second user utterances among one another when controlling the surgical visualization system.

If multiple second user utterances are captured, then these are in a relationship not only with the first user utterance, but also with one another. The time differences or intervals between successive second user utterances may vary, and may likewise be used for the control.

By way of example, it is possible to take into account a sequence of the second user utterances within the period of time. If no second user utterances occur over a valid time window, this may likewise be a parameter for the control.

It is also possible to recognize more complex temporal patterns over the entire captured sequence of second user utterances and process them with reference to the time interval.

In some examples, the temporal relationship between all captured second user utterances may be taken into account for the control, for example in order to select at least one second user utterance and/or in order to exclude at least one second user utterance.

Determining a user input using the captured user utterance may be done in various ways. One possibility is a rule-based approach in which predefined rules are used to recognize particular features or patterns in the user utterance and to derive the corresponding user input therefrom. By way of example, certain keywords or gestures could be permanently linked to specific inputs. Further conceivable approaches comprise, inter alia, pattern recognition, statistical models, knowledge-based systems, or multimodal fusion, wherein information from different modalities of a first user utterance (for example speech and gestures) is combined in order to improve the input recognition.

Another possibility for determining a user input is the use of machine learning methods, in particular neural networks, that is to say using machine learning models. In this case, the system is trained, on the basis of training data, to learn the assignment between user utterances and user inputs. After training, the system may then generate inputs matching new user utterances. Neural networks are also capable of recognizing complex user inputs in the data, for example based on speech recognition.

Controlling the surgical visualization system may comprise triggering an action of the surgical visualization system. The type of action may be specified by the first user input. The at least one second user input may trigger and/or parameterize the action.

Thus, while the first user input specifies the type of action (for example “zoom”), the second user input may initiate or trigger the performance of the action or specify a context for this action. The triggering increases safety in relation to incorrect operations (“two-factor control”). The parameterization allows the action to be implemented more accurately.

Typically, the information content in the first user input is greater than the information content in the second user input. This is due to the fact that the first user input typically has to select the type of action from a relatively large candidate space: a large number of actions to be performed are often available. On the other hand, the second user input merely has to confirm the performance of the particular action or set it within a limited parameter space, wherein such an input often has lower information content (for example, clicking a button or saying “OK!” could be sufficient). This discrepancy in the information content is one possible reason why a single first user utterance takes place during the period of time, while a large number of second user utterances take place.

The multiplicity of second user inputs may define coordinates in a continuous space, wherein the coordinates have a development (or progression) during the period of time. The method may furthermore comprise applying a filter, in particular a low-pass filter or a Kalman filter, to the coordinates, thereby smoothing the development (progression).

In some examples, the second user inputs may comprise second user inputs of different second user utterance types, for example the gaze direction of the user or the gaze position of the user on the display, or the position of the instrument tip. Such second user utterances provide information about the current region of interest of the user, which may be characterized by a target position (POI), or a target region (ROI), of the user in the operating region, but said information may contain noise and inaccuracies.

To obtain a more robust estimate of the POI, a low-pass filter may be used in some examples.

In order to obtain a more robust estimate of the POI, it is also possible to use a Kalman filter that combines the information from both signals, that is to say both types of second user utterances. The Kalman filter is able to estimate the most probable POI taking into account the uncertainties of the individual measurements and the dynamics of the POI over time. To this end, it is possible to define a state model that describes the position and possibly also the speed of the POI in space. The two types of second user utterances are modelled as observations with corresponding uncertainties. A combination of the data from two different second user utterance types is able to provide a more accurate and more stable estimate of the POI than would be possible with each signal alone.

It may be advantageous to take into account at least three, or at least four, or more different second user utterance types.

A filter parameter of the filter may depend on a type of content displayed on a display of the surgical visualization system, and/or a phase of a surgical workflow, and/or the first user utterance type, and/or the second user utterance type and/or a type of the first user input that was determined based on the first user utterance.

A surgical visualization system is configured to perform any method or any combination of methods according to the present disclosure. The method may be performed for example by a control unit, that is to say a shared or dedicated computing device of the surgical visualization system.

A control unit of a surgical visualization system is configured to perform any method or any combination of methods according to the present disclosure.

The control unit comprises a processor, memories and an interface for receiving and providing sensor signals and/or user utterances, wherein the memory unit comprises instructions that are able to be executed by the computing unit and that, when they are executed by the computing unit, cause said computing unit to perform the steps of any method or any combination of methods according to the present disclosure.

A computer program or computer program product comprises instructions that, when they are executed by a processor, cause said processor to perform the steps of any method or any combination of methods according to the present disclosure.

The described techniques are able to control a surgical visualization system so as to process and present image data based on user utterances, and are thus able to provide imaging assistance for a surgical procedure, for example targeted navigation in an image dataset, such as for example a 2D or 3D image dataset of a person, in order to present a target region, but comprise or require no steps of a surgical procedure themselves. The described techniques are based on capturing and processing user utterances in order to determine control commands for modifying a presentation of a surgical visualization system, wherein these interactions between a user and the surgical visualization system may also be performed for example before the start of a surgical procedure or after the end of a surgical procedure.

Although the features described in the above summary and the following detailed description are described in association with specific examples, it should be understood that the features may be used not only in the respective combinations, but also in isolation or in any desired combinations, and features from different examples may be combined with one another and thus correlate with one another, unless expressly indicated otherwise.

The above summary is therefore intended to give only a brief overview of some features of some exemplary embodiments and implementations and should not be understood as a restriction. Other embodiments may comprise features other than those described above.

### BRIEF DESCRIPTION OF THE FIGURES

The invention is explained in more detail below on the basis of preferred exemplary embodiments with reference to the accompanying drawings, wherein identical reference signs denote identical or similar elements. The figures are schematic illustrations of various exemplary embodiments of the invention, wherein the elements illustrated in the figures are not necessarily illustrated as true to scale. Rather, the various elements illustrated in the figures are rendered in such a way that their function and general purpose become comprehensible to a person skilled in the art.

FIG. 1 schematically illustrates a surgical visualization system in accordance with various examples.

FIG. 2 schematically illustrates a temporal profile of voice command recognition and a change in gaze position over multiple phases, in accordance with various examples.

FIG. 3 is a flowchart of one exemplary method.

The properties, features and advantages of this invention described above and the way in which they are achieved will become clearer and more clearly understood in association with the following description of the exemplary embodiments, which are explained in greater detail in association with the drawings.

It should be noted here that the description of the exemplary embodiments should not be understood in a limiting sense. The scope of the invention is not intended to be restricted by the exemplary embodiments described below or by the figures, which serve merely for illustration.

### DETAILED DESCRIPTION

The present invention is explained in greater detail below on the basis of preferred embodiments with reference to the drawings. Connections and couplings between functional units and elements illustrated in the figures may also be implemented as an indirect connection or coupling. A connection or coupling may be implemented in a wired or wireless manner. Functional units may be implemented as hardware, software or a combination of hardware and software.

Various techniques for a surgical visualization system will be described. However, it should be understood that the described techniques are applicable to any system in which control or manipulation is carried out based on multimodal user inputs having different durations and frequencies. The techniques are thus not restricted to the surgical context, but may be used in a wide variety of human-machine interfaces, for example when interacting with computers, robots, vehicles or other technical systems. The method according to the invention offers a general framework for the processing and fusion of user inputs with different temporal properties.

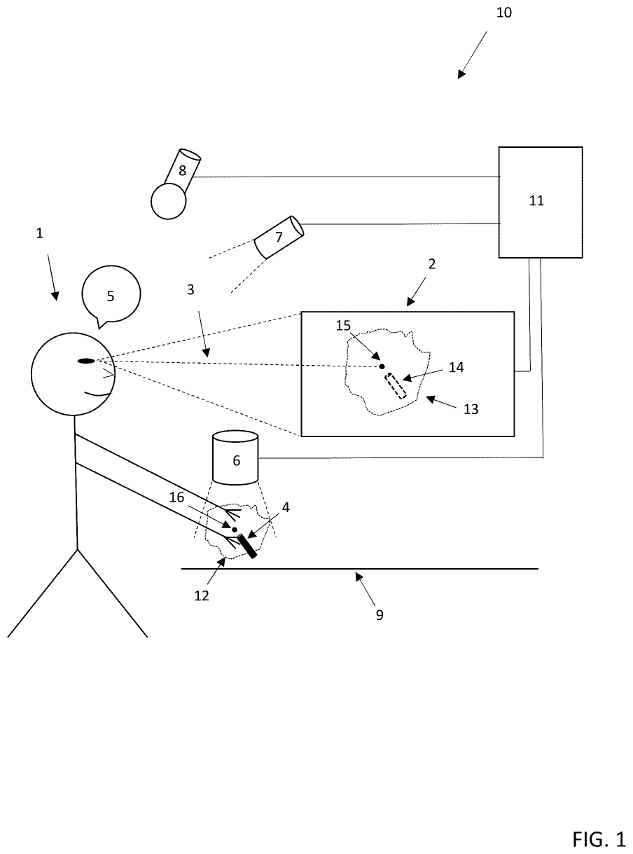

FIG. 1 schematically illustrates a surgical visualization system 10 in accordance with various examples.

As may be seen in FIG. 1, the surgical visualization system 10 comprises a control device 11 that controls the individual components of the system, in particular a presentation of an operating region 13 on a display 2 of the system. The display in FIG. 1 is an external display, but it would also be possible to present the image in optical eyepieces, as an image on a 3D monitor, as an image in digital eyepieces, or as an image in AR glasses to the user 1.

A patient or an object to be examined may be arranged on an operating table 9, said object comprising an operating region and being captured by the imaging system 6 and being presented on a display 2 by the visualization system 10. In this example, the imaging system 6 comprises a surgical microscope (OPMI) for magnified imaging of the operating region 12.

The surgical visualization system 10 is controlled based on multimodal user inputs 3, 5 of different user utterance types having different durations and at different times.

A user 1 of the surgical visualization system 10 operates a surgical instrument 4. The surgical instrument 4 is arranged within an operating region 12. The operating region 12 is presented to the user 1 on the display 2 as an imaged operating region 13 by the surgical visualization system 10 by way of an imaging system. The operating region 12 is arranged in a field of view of the surgical visualization system 10. Furthermore, the surgical instrument 4 is also imaged by the surgical visualization system 10 and presented to the user 1 on the display 2 as an imaged surgical instrument 14. The imaging system may comprise a camera 6, for example.

To control the surgical visualization system, the user interacts with the system 10 using various modalities.

One of these modalities is the linguistic user utterance 5, which in this case represents a first user utterance, and is used for voice control of the system. The linguistic user utterance is captured using a microphone 8 and defines a desired control action, such as for example “focus this region”. The linguistic utterance 5 extends over the duration of a time interval in a longer (reference) period of time. From the linguistic utterance, processing carried out by the control device using speech recognition is used to determine a voice command indicating the desired control action for the surgical visualization system 10.

Examples of voice commands may be “Hey, go there”—the camera should for example perform a linear translational movement in order to centre the POI in the field of view, “look”—the camera should tilt about its optical midpoint and centre the POI in the field of view, or “autofocus there”—the autofocus algorithm should focus pixels in the vicinity of the POI.

At least partially in parallel with the capturing of the linguistic utterance 5 and the determination of the voice command or the control action, a multiplicity of second user utterances of different second user utterance types are captured.

A first second user utterance type is the gaze direction of the user 3 in relation to the presentation of the operating region relative to the display 2. The gaze direction is captured and tracked using an eye tracking system comprising a camera 7. It would also be possible to capture and track an orientation, in particular a forward direction, of the head of the user 1 using the camera 7.

From the gaze direction 3, as a second user utterance, it is possible to use the known spatial setup of the surgical visualization system 10 to determine a gaze position 15 on the display 2, and also to determine therefrom, based on the imaging parameters of the system, as a user input, a target point 16 (or point of interest, POI), or in turn therefrom a target region (region of interest, ROI), with respect to the operating region 12 or with respect to the presented image data of the operating region 12. The gaze position 15 on the display 2 thus corresponds to a POI in the field of view of the OPMI 16, wherein this correspondence may easily be calculated by a person skilled in the art.

A further second user utterance type is the position of a surgical instrument 4 with respect to the operating region 12, said position being determined using the imaging system 6b and also imaging the operating region. The POI 16 in the operating region 12 may also be determined from tracking the position and/or orientation of the surgical instrument 4.

The gaze direction data and instrument position data are acquired repeatedly over the period of time and buffered together with associated timestamps (as an example of time information). These are used to track the POI 16 of the user 1 in the operating region 12.

Both the gaze direction and the positions of the surgical instrument 4 may thus be captured and processed as second user utterances in order to determine a target point (POI) or target region (ROI) of the user 1 in the operating region 12.

The surgical visualization system 10 and the control device 11 are designed to perform any method or any combination of methods according to the present disclosure.

In this example, the buffered POIs 16 of both input modalities are filtered and merged, resulting in a robust and smooth estimate of the POI 16 of the user. The POIs 16 may be present (as data points) in a timeseries and/or stored or buffered.

The merged POIs are stored by the control device 11 with respective time information in a buffer data structure, and thus represent sequences of data points of the respective second user utterances.

A control command for the surgical visualization system is generated based on the determined voice command and the buffered POI data. This command is used to adapt the visualization in accordance with the user's intention, for example by focusing on the specified target region. The result is displayed to the surgeon on the display.

The control may comprise for example controlling the OPMI 6, such as for example the XY movement by moving the OPMI 6 linearly in translation, rotating the OPMI camera, digitally cropping the image region, or an autofocus setting at a particular position in the operating region 12.

However, the eye tracking signal may be noisy, for example due to subconscious gaze flickering of the user, and may be slightly distorted, for example if the user is distracted and is looking elsewhere than the intended gaze position. In this case, the use of raw unfiltered gaze positions as POI is unreliable.

Moreover, the gaze position changes over time. When eye tracking is used together with voice command recognition, there is a duration of the spoken sentence and a delay in terms of speech recognition. During this time period, the user's gaze position may have already changed.

To address these challenges, the gaze position data and instrument position data are acquired repeatedly over a period of time and stored in a buffer data structure together with time information. Filtering, merging and taking into account the time information of the buffered POIs of both input modalities makes it possible to achieve a more robust and smoother estimate of the actual point of interest of the user in the operating region.

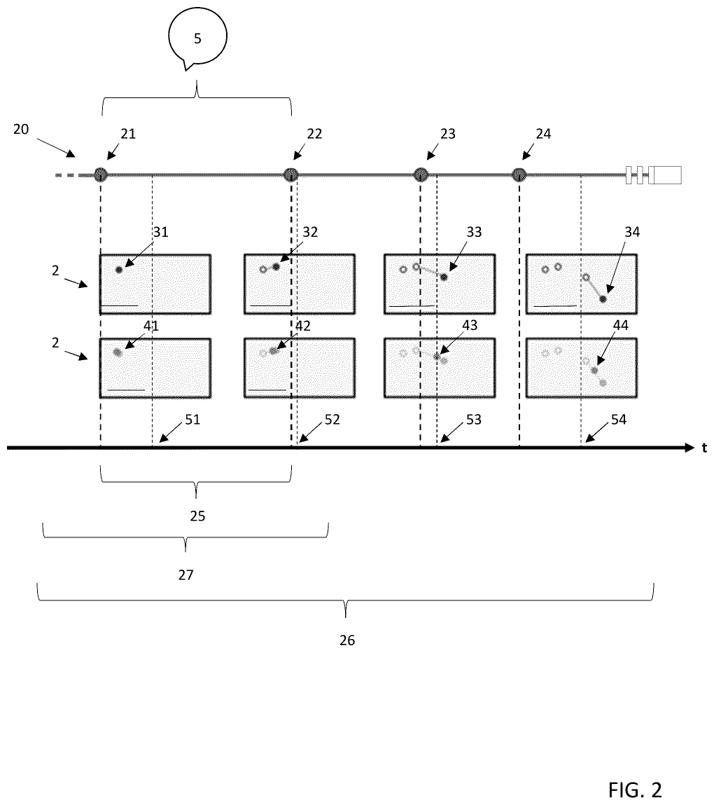

The control process takes into account a temporal correlation between the individual gaze and instrument data points and the voice command. The time interval of the speech input is incorporated here in order to select, from the timeseries of buffered POIs, only the POIs relevant to the voice command for the control. This improves reliability and control, as will be described in more detail with reference to FIG. 2.

FIG. 2 schematically illustrates a temporal profile of a voice control process 20 and a change in the raw gaze positions 31-34 and filtered gaze positions 41-44 over multiple phases of the voice command recognition, in accordance with various examples.

The top row in FIG. 2 illustrates the temporal profile of a voice control process 20 divided into different phases with respect to the linguistic utterance 5 (first user utterance) of FIG. 1.

Time 21 marks the start of the linguistic utterance 5, for example a spoken sentence or sentence fragment, made by the user. At time 22, the linguistic utterance ends. The time interval between the times 21 and 22 thus corresponds to the time interval 25 over which the linguistic utterance extends and during which the linguistic utterance 5 is captured. The time interval 25 lies in a longer period of time 26, which may comprise times before and/or after the time interval 25.

Between times 22 and 23, the captured linguistic utterance 5 is processed (speech recognition) in order to determine therefrom a voice command or user intention (corresponding to a first user input). At time 23, the speech recognition is complete and the voice command has been recognized.

Between time 23 and time 24, a desired control action of the surgical visualization system 10 may be determined based on the voice command; for example, the control action may be parameterized.

Based on the recognized voice command, a control action of the surgical visualization system 10 is initiated and subsequently performed based on the voice command with a time delay starting from the time 24; for example, the control action is activated or triggered at the time 24.

FIG. 2 illustrates, in the middle and bottom rows, changes in the user's gaze positions 31-34 and 41-44 (second user utterance of a second user utterance type “gaze direction”) during the voice control phases. The second user utterances are illustrated here during the time interval 25; they may also lie at least partially before and/or after the time interval 25 in the period of time 26.

The middle row of FIG. 2 shows changes in the raw, that is to say unprocessed or unfiltered gaze positions 31-34 of the user, associated with respective times 51-54 at which the respective unfiltered gaze positions were acquired. Each gaze position 31-34 represents a measurement at a particular time. Generally speaking, time information may be associated with each of the gaze positions, which may for example represent a respective time or a respective duration with which the respective gaze positions may be associated.

The bottom row of FIG. 2 shows changes in the filtered gaze positions 41-44 of the user, associated with the respective times 51-54, wherein the filtered gaze positions 41-44 are determined from the unfiltered gaze positions 31-34 by filtering. In the example of FIG. 2, the times of the filtered gaze positions correspond to those of the unfiltered gaze positions 32-34, but other times for the filtered gaze positions 41-44 could also be determined from the times 51-54 through a corresponding filtering operation. Each gaze position 32-34 and 42-44 is thus associated with time information indicating when the respective gaze positions were captured.

In this case, the gaze position is captured by the eye tracking system 7. Each point represents a gaze measurement at a particular time. It may be seen that these measurements are distributed over the display and also vary during the voice command.

As may be seen in FIG. 2, noise and variability are reduced at the filtered gaze positions 41-44 by applying a filter. The filtered gaze positions are more concentrated on relevant regions of the display 2.

The present disclosure is based on the finding that the gaze positions 33, 34, 43 and 44 that come after recognition of the voice command 23 are less relevant to the performance of the control action than the gaze positions 31, 41, and possibly also gaze positions 32 and 42, due to their temporal proximity to the linguistic utterance. There is a time delay between the end 22 of the linguistic utterance 5 and the activation of the command 24 by the control unit 10. During this delay, the gaze positions continue to change. Since the gaze positions change continuously, in particular even after the end of the voice command as well, a temporal correlation is crucial for determining the relevant gaze position.

By way of example, it may be the case that, although the user is looking at the target position and starts to say the linguistic utterance (for example “move to this position” or “autofocus the view there”), they already look away before the entire sentence has finished. A further example would be if the surgeon continues to look at the target position until the entire sentence has finished, but the voice recognition algorithm needs one second to recognize the command and to activate the movement, at which time the surgeon is already looking away. In such situations, the use of the last-determined gaze position as POI is unreliable when the voice command is activated.

Simply using the most recently captured gaze position when activating the control action is thus problematic if the user is already looking at another region of the display 2 at this time 24. Instead, it is advantageous to select the gaze position to be used taking into account the temporal context of the voice command and the gaze positions. It is also possible to carry out a different form of prioritizing the different gaze positions with respect to one another—for example a relative weighting.

By way of example, the gaze positions may be stored together with time stamps in a buffer data structure. When the voice command is activated, the relevant gaze position 3 may then be selected on the basis of the temporal relationship between the buffered gaze positions 3 and the time interval of the voice command 5, even if this does not coincide with the end time of the voice command 5. This combination of the modalities taking into account their temporal correlations achieves robust and reliable control of the surgical visualization system.

From the aspect of filtering, for example, a low-pass filter may be applied to the individual measurements of the second user utterances in order to smooth the target position. Non-limiting examples of such filtering comprise for example a simple PT1 low-pass filter, sensor fusion or a general combination of various measurements for determining the POI (eye tracking, head-forward direction, position of the surgical instrument in the image), a Kalman filter, dynamic filtering based on the phases of a voice command, or filtering the POI, which is dependent on the image content and the surgical phase.

In some examples, a low-pass-like filter may be applied in order to smooth the gaze positions, or else the target positions (POI). This filter may be implemented in various ways in order to smooth the temporal development of the gaze points, that is to say the gaze point trajectory, or POI trajectory.

In some examples, a simple PT1 low-pass filter may be used. This first-degree filter is able to perform weighted averaging of the positions over time, wherein the influence of older measurements may decay exponentially. Selecting a suitable time constant makes it possible to adapt the smoothing effect to the dynamics of the gaze movements.

In some examples, it is possible to apply sensor fusion, in which second user utterances from two or different second user utterance types may be combined in order to determine the target positions (POI). For this purpose, for example, it is possible to use data from the eye tracking, the head-forward direction and the position of the surgical instrument in the image. Combining this information makes it possible to achieve a more robust estimate of the POI, which may be less susceptible to disturbances or inaccuracies in individual modalities.

In some examples, it is possible to use a Kalman filter to smooth the gaze positions or target positions. The Kalman filter is a recursive algorithm that is able to estimate the state of a system from noisy measurements. In the present case, the state may comprise the position and speed of the POI.

In some examples, the target position may be filtered dynamically based on the phases of the voice control. In this case, for example, the gaze positions may be weighted to a greater extent if a sentence is currently being spoken, since, in this phase, the gaze position may be particularly relevant to the interpretation of the user intention. By contrast, the gaze positions may be weighted to a lesser extent during the delay in speech recognition after the end of the sentence, since, in this phase, the user may possibly already be looking at a different image region.

In some examples, the target positions may also be filtered depending on the image content currently being presented and the surgical phase. Thus, for example, in phases in which particularly fine structures are able to be seen in the image, the filter time constants may be reduced in order to enable a higher resolution of the gaze positions. In phases with coarser structures or in overviews, on the other hand, longer time constants may be used in order to achieve greater smoothing. The type of surgical intervention and the typical gaze movement patterns associated therewith may also be taken into account when selecting the filter parameters.

Filtering the gaze positions or target positions in order to determine a smoothed timeseries of POIs may thus improve the reliability and accuracy of the control.

In order to increase the reliability and user-friendliness of the POI estimate using gaze movements and thus improve user experience, buffering is performed for the raw gaze positions 31-34 and/or the filtered gaze positions 42-44.

The trajectory of the gaze positions over time may be buffered so that the system is able to retrieve gaze positions from earlier phases of the voice control when the voice command is activated.

For this purpose, the determined unfiltered gaze positions 31-34, or the filtered gaze positions 41-44, are buffered in a buffer data structure (for example a first-in-first-out buffer data structure) for a certain time period (for example 10 seconds). The system stores the timestamps in association with the buffered gaze positions in the buffer data structure. The system likewise obtains time information characterizing the time interval, for example the start and end time of the time interval. When the linguistic utterance has been recognized as a valid voice command, the system selects one or more gaze positions relevant to the voice command from the buffered gaze directions and uses them to control the surgical visualization system.

In the example of FIG. 2, for example, the gaze directions 31 and 41 that were captured at the same time as the first user utterance, and/or the gaze directions 31, 32 and 41, 42 within a valid time window 27 around the start or the end of the time interval 25, may be used for the control.

There are various possibilities as to how the buffer data structure may be implemented. By way of example, the buffer data structure may store the raw or filtered eye tracking positions, or gaze positions, or head-forward direction, or else the target positions (POI) determined therefrom in the image dataset or in the operating region. The buffering may store for example data points at the system frequency, or half the system frequency, or at fixed time variants, or at a dynamic sampling rate or at fixed sampling variants. Furthermore, the user or an Al algorithm may configure the system such that it uses such a buffered element that best corresponds to the time interval. By way of example, by comparing the timestamps with the time when the linguistic utterance begins, or when the linguistic utterance ends, or a time in between, or with a fixed time window relative to the time interval of the voice command.

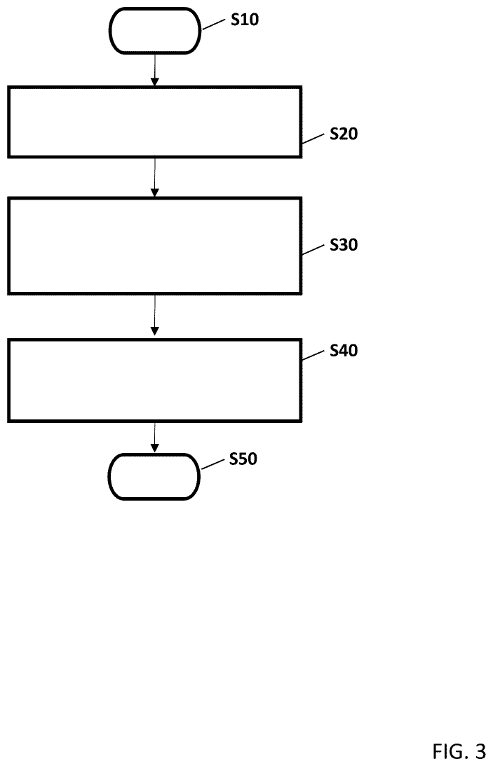

FIG. 3 is a flowchart of one exemplary method for controlling a surgical visualization system.

The method begins in step S10.

In step S20, a first user utterance of a first user utterance type is obtained. The first user utterance extends over a time interval within a period of time.

In step S30, a multiplicity of second user utterances of at least one second different user utterance type are obtained. The second user utterances are arranged in a manner distributed within the period of time and comprise different second user utterances. In step S30, time information is also obtained for each of the multiplicity of second user utterances.

The data obtained in step S30 are buffered, for example in a buffer memory with a FIFO structure.

In step S35, temporal relationships between each of the multiplicity of second user utterances and the first user utterance are determined. This may be carried out for example based on a comparison between timestamps and the time interval in which the first user utterance takes place.

In step S37, at least one of the second user utterances is then prioritized based on the temporal relationships. By way of example, such prioritization may comprise selecting the at least one second user utterance and discarding the non-selected one or more second user utterances. Such prioritization could also comprise setting weights in connection with the at least one second user utterance to comparatively higher values.

In step S40, the surgical visualization system is controlled based on the first user utterance, the prioritized at least one second user utterance and a temporal relationship between the first user utterance and the at least one second user utterance.

The method ends in step S50.

### LIST OF REFERENCE SIGNS

1 User
2 Display
3 Second user utterance-gaze direction
4 Surgical instrument
5 First user utterance-linguistic utterance
6 Imaging system
7 Eye tracking system
8 Microphone
9 Examination table
10 Surgical visualization system
11 Control device
12 Operating region
13 Presented operating region
14 Presented surgical instrument
15 Gaze position
16 Target point (point of interest, POI)
20 Voice control phases
21 Time: start of the first user utterance
22 Time: end of the first user utterance
23 Time: voice command recognized
24 Time: start of the control action
25 Time interval
26 Period of time
27 Valid time window
31-33 Unfiltered gaze positions
41-44 Filtered gaze positions
51-54 Times associated with gaze positions
S10 to S50 Method steps

1. A computer-implemented method for controlling a surgical visualization system, the method comprising:
obtaining a first user utterance of a first user utterance type, wherein the first user utterance extends over a time interval within a period of time;
obtaining and buffering a multiplicity of second user utterances of at least one second user utterance type and respective associated time information for each of the multiplicity of second user utterances, wherein the second user utterances are distributed within the period of time and comprise different second user utterances of each of the at least one second user utterance type;
determining temporal relationships between each of the multiplicity of second user utterances and the first user utterance based on the time information and the time interval,
prioritizing at least one second user utterance from the multiplicity of second user utterances based on the temporal relationships, and
controlling the surgical visualization system based on the first user utterance and the prioritized at least one second user utterance.

2. The computer-implemented method according to claim 1, the method further comprising:
for each second user utterance of the multiplicity of second user utterances: carrying out a check to determine whether the respective temporal relationship satisfies one or more predefined check criteria,
wherein the at least one second user utterance is prioritized depending on a result of the checks to determine whether the temporal relationships satisfy the one or more check criteria.

3. The computer-implemented method according to claim 2, wherein the one or more check criteria comprise a check to determine whether a point in time associated with a corresponding second user utterance and indicated by the time information lies in a particular time range before the end of the time interval.

4. The computer-implemented method according to claim 1, the method further comprising:
determining a first user input based on the first user utterance; and
determining at least one second user input for the prioritized at least one second user utterance,
wherein the surgical visualization system is controlled based on the first user input and the at least one second user input.

5. The computer-implemented method according to claim 4,
wherein controlling the surgical visualization system comprises triggering an action of the surgical visualization system,
wherein a type of the action is specified by the first user input, and
wherein the at least one second user input triggers and/or parameterizes the action.

6. The computer-implemented method according to claim 1,
wherein the multiplicity of second user utterances defines coordinates in a continuous space, wherein the coordinates have a development during the period of time,
wherein the method further comprises:
applying a filter to the coordinates, in particular a low-pass filter or a Kalman filter, thereby smoothing the development.

7. The computer-implemented method according to claim 6, wherein a filter parameter of the filter depends on a type of content displayed on a display screen of the surgical visualization system, a phase of a surgical workflow and/or the first user utterance type.

8. The computer-implemented method according to claim 1, wherein several of the multiplicity of second user utterances are different from one another and lie in the time interval.

9. The computer-implemented method according to claim 1, wherein each of the second user utterances extends over a respective duration, wherein each of the durations is shorter than the time interval over which the first user utterance extends, wherein preferably a length of each of the durations is less than 50% of the length of the time interval over which the first user utterance extends, particularly preferably less than 10%.

10. The computer-implemented method according to claim 1, wherein the first user utterance comprises one of the following:
a linguistic utterance made by the user;
a gesture made by a body part of the user;
a touch gesture on a touch interface;
a brain/computer interface signal; or
a multimodal combination of the above.

11. The computer-implemented method according to claim 1, wherein the second user utterances comprise pointing the user towards a target region in a field of view of the surgical visualization system.

12. The computer-implemented method according to claim 1, wherein the second user utterances comprise one of the following:
a position and/or orientation of at least one body part of the user;
a gaze direction of a user; and
a position and/or orientation of a surgical instrument operated by the user, or
a multimodal combination of the above.

13. The computer-implemented method according to claim 4,
wherein the first user utterance comprises a linguistic utterance made by the user, wherein the second user utterances comprise at least one first group of second user utterances comprising gaze directions of the user, and wherein the second user utterances comprise at least one second group of second user utterances comprising a position and/or orientation of a surgical instrument operated by the user, and
wherein the at least one second user input is determined using at least in each case a second user utterance from the first and second group of second user utterances.

14. A control unit for a surgical visualization system, the control unit configured to carry out the method according to claim 1.

15. A surgical visualization system comprising a control unit according to claim 14.
